(19) United States
(12) Patent Application Publication
(10) Pub. No.: US 2005/0269231 A1
(43) Pub. Date: Dec. 8, 2005

(54) Container for packaging and deploying devices

(76) Inventors: Jack White, Alpharetta, GA (US); Stephen N. Carr, Flowery Branch, GA (US)

Correspondence Address: Kevin J. Dunleavy;KNOBLE, YOSHIDA & DUNLEAVY, LLC, Eight Penn Center, Suite 1350, 1628 John F. Kennedy Boulevard, Philadelphia, PA, 19103, US

(21) Appl. No.: 10/863,186

(22) Filed: Jun. 8, 2004

(51) Int. Cl. A61B 17/06

(52) U.S. Cl. 206/438

(57) ABSTRACT

A shipping container for medical devices that uses part of the container to transfer the device from the container to another location after the container is opened. A container is provided which has a first container portion and a second container portion. One portion of the container is provided with a grip that is graspable by a user's hand and a holder for holding the device using that same portion of the container to permit that portion of the container to be gripped and used to transfer the device from the container to another location without having to directly contact the device. The portion of the container used for transferring the device serves as a barrier between the device and the user to minimize contamination of the device by the user or to minimize contamination of the user by the device. Also described is a method for storage and deployment of a packaged device using the container of the invention.

### BACKGROUND OF THE INVENTION

1. Field of the Invention

The invention relates to the field of containers for packaging devices. Specifically the invention relates to a container that can be employed to grasp and transfer a packaged device from the container to another location in order to minimize contamination of the device prior to use.

2. Description of the Related Technology

It is desirable to keep certain types of devices sterile or to minimize contamination of such devices until used. Exemplary devices can include probes, knives, scissors, cutters, applicators, implantation devices, catheters and other devices for which sterility or at least minimal contamination is required. Typically, these devices are sealed in a package and sterilized after sealing by treatment with radiation or gas. An exemplary package for this type of device consists of a thermoformed plastic blister tray provided with an airtight seal formed by a spun polyester sheet. Other packages are formed from two mating plastic trays that snap together with the device inside, the entire assembly being sealed in an air-tight pouch.

While such packages are effective for delivering the devices to the location of use in a substantially uncontaminated state, there is an additional need to minimize contamination of such devices that occurs during transfer of the device from the package to another location for use, thereby increasing the ease with which a device may be used. For example, during the course of surgery, it is often necessary to remove various instruments and devices from sterile packaging. Medical personnel are required to maintain an environment of sterility and need to be dressed accordingly. Gloves worn by medical personnel to maintain sterility often make it difficult to manipulate items in packaging. Current packaging methods and containers do not sufficiently address the need to maintain sterility and to provide ease of transport of the device upon opening the package. Specifically, current packages generally do not facilitate the transfer of the sterile contents of a package into a sterile field without contamination or damage to the contents by, for example, having to dump the contents onto a table for use.

For example, U.S. Pat. No. 5,842,567 to Rowe et al. discloses a quick release package for use with medical devices. The package is made of plastic. The package is constructed so that it is form fitted around a medical probe. The package has snaps for closing the package. The package is designed primarily for quick opening by pressing down on the edge of a tab. U.S. Pat. No. 5,178,282 to Williams discloses a modular surgical packaging system. The container allows the stored components to be sterilized.

U.S. Pat. No. 6,012,580 to Peters et al. discloses a package for medical implants. The package may be squeezed from the sides to release the medical device contained therein. This permits removal of the medical device without touching the device and risking contamination. However, the primary purpose of the finger holds used on the package is to provide a means for opening the package and not a means for gripping the device housed in the package using the package itself as a gripping means. This increases the risk of accidentally dropping the device and therefore still contaminating the device despite the release mechanism disclosed.

Therefore there remains a need in the field for a container that keeps packaged device sterile and permits the handler of the object to easily transfer the object from the container to its place of use, while minimizing potential contamination of the device.

There is also a need for packages which can be used to permit a non-sterile worker to transfer the sterile contents of the package into a sterile field without a significant risk of damage or contamination to the contents of the package.

Also, there is a need for packages which can be used to package and ship potentially hazardous devices which may include, for example, corrosive materials, poisonous materials, irritants and/or radioactive materials, in a safe manner and permit the end user to handle such hazardous devices with a minimum of contamination of the user by the device.

### SUMMARY OF THE INVENTION

Accordingly, it is an object of certain embodiments of the invention to provide an improved container for packaging various types of devices. The present invention provides a container useful for both packaging a device and for transferring the device from the container to its place of use with a minimum amount of contamination of the device by the user, or with a minimum amount of contamination of the user by the device.

A container is provided which has a first container portion and a second container portion. One portion of the container is provided with a grip that is graspable by a user's hand and a holder for holding the device using that same portion of the container to permit that portion of the container to be gripped and used to transfer the device from the container to another location without having to directly contact the device. The portion of the container used for transferring the device serves as a barrier between the device and the user to minimize contamination of the device by the user or to minimize contamination of the user by the device.

In a second aspect, the present invention provides a method for shipping and handling packaged devices that minimizes contamination of the devices during shipping and transfer of the device out of the shipping container for use. In the method, a container is provided having first and second container portions. One portion of the container is provided with a grip that is graspable by a user's hand and a holder for holding the device using that portion of the container. In the method, the device is placed into the container, and the container is sealed and sterilized. Then, upon opening the container, the user grasps the grip that forms part of one portion of the container and removes the device from the container using that portion of the container as a barrier between the user and the device. The device is held by the portion of the container provided with the grip to thereby permit removal of the device from the container without directly contacting the device.

For a better understanding of the invention, its advantages, and the objects obtained by its use, reference should be made to the drawings which form a further part hereof, and to the accompanying descriptive matter, in which there is illustrated and described preferred embodiments of the invention.

### BRIEF DESCRIPTION OF THE DRAWINGS

FIG. 1 is a side view of one embodiment of a container in accordance with the present invention.

FIG. 2 is rear view of the first container portion of the container of FIG. 1.

FIG. 3 is a front view of the first container portion of the container of FIG. 1.

FIG. 4 is a view of the top of the first container portion of the container of FIG. 1.

FIG. 5 is a of the bottom of the first container portion of the container of FIG. 1.

FIG. 6 is a top view of the second container portion of the container shown in FIG. 1.

FIG. 7 is a bottom view of the second container portion of the container shown in FIG. 1.

FIG. 8 depicts an ocular brachytherapy device for which the container of FIG. 7 is specially designed.

### DETAILED DESCRIPTION OF THE PREFERRED EMBODIMENT(S)

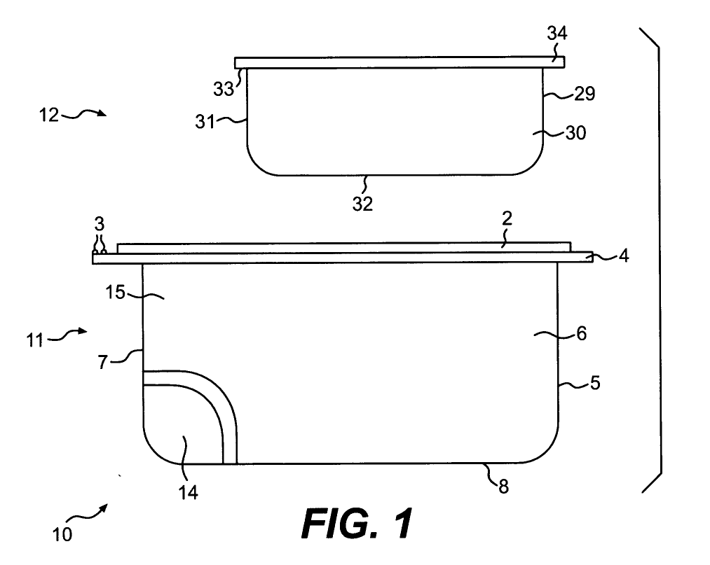

Referring now to the drawings, wherein like reference numerals designate corresponding structure throughout the several views, FIG. 1, shows a view of container 10 from the side. Container 10 includes a first container portion 11 and a second container portion 12. Container 10 is designed for storage and shipment of a device to a location where the device will be used. Container 10 is also designed to facilitate removal and transfer of the packaged device from container 10 to another location with a minimum amount of contamination of the device by the user or with a minimum amount of contamination of the user by the device, by providing a second container portion 12 that can be employed to grasp and transfer the packaged device from container 10 to another location, without requiring the user to touch the device. The embodiment of the invention illustrated below is designed for use with an ocular brachytherapy device for which sterility of the device for use is required. However, the present invention may be employed for use with a variety of other devices as well.

Specifically, the container 10 of the present invention is particular useful for the packaging and shipping of sterile devices. The container 10 of the present invention permits the user of the sterile device to open the container, and deploy the sterile contents of the container with a minimal risk of damage or contamination to the contents of the container 10. In fact, a non-sterile worker can open the container 10 and deploy the sterile contents with a minimal risk of contamination and without risk of damage which can be caused by, for example, having to dump the contents of the container onto a table or other surface. Instead, the sterile contents of container 10 can be directly deployed, by a non-sterile worker, into a sterile field for immediate use in a sterile environment.

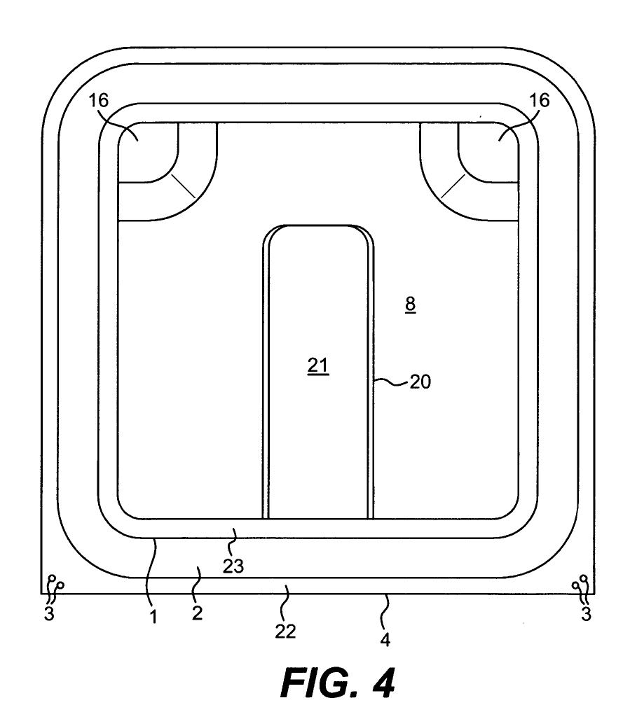

First container portion 11 is comprised of a bottom 8 two sides 6, a front 5 and a back 7 which together form a receptacle capable of receiving at least a portion of second container portion 12, as well as the packaged device therein. First container portion 11 includes a pair of corner indentations 14 in the corners between bottom 8, sides 6 and back 7 which form a pair of shoulders 16 inside of first container portion 11, as shown in FIG. 4. Corner indentations 14 may also be designed to provide a convenient location to grasp first container portion 11 of container 10 between two fingers of one hand when opening or closing container 10.

Second container portion 12 fits within first container portion 11 between front 5 and shoulders 16 to create a space in the area 15 of first container portion 11. The space in the area 15 of first container portion 11 is desirable in this embodiment of the invention since the packaged device is designed for attachment to an articulating arm. Thus, the space in area 15 provides room for an attachment portion of the packaged device to extend beyond the periphery of second container portion 12 when the packaged device is located in container 10. Due to this feature, the packaged device can be attached directly to the articulating arm while it is still held by second container portion 12 because the attachment portion of the packaged device extends sufficiently far out of second container portion 12 that it can be attached to an articulating arm without interference from second container portion 12.

Accordingly, in one aspect of the invention, second container portion 12 may be sized relative to the packaged device to permit a portion of the packaged device to extend beyond the periphery of second container portion 12 to facilitate interaction of the packaged device with another device without having to remove the packaged device from second container portion 12 or having to directly handle the packaged device. First container portion 11 may be sized appropriately to accommodate the portion of the packaged device that extends beyond the periphery of second container portion 12, as well as at least part, or all, of second container portion 12, as desired.

Shoulders 16, sides 6 and front 5 of first container portion 11 together limit the movement of second container portion 12 relative to first container portion 11, when second container portion 12 is inserted within first container portion 11. It is possible to size first and second container portions 11, 12 such that the walls of second container portion 12 abut with shoulders 16 when second container portion 12 is located within first container portion 11, but this is not necessary. Thus, it is also possible to leave some space between the walls of second container portion 12 and shoulders 16 to permit a certain amount of play between the first and second container portions 11, 12 when they are associated with one another. In general, it is desirable to minimize the amount of force required to separate first and second container portions 11, 12 to facilitate opening container 10. For this reason, it may be desirable to permit a certain amount of play between first and second container portions 11, 12 when they are associated with one another. Also, due to the nature of the packaged devices, it will generally be necessary to seal container 10, by, for example, shrink-wrapping container 10, or heat-sealing using a Tyvek® lid or pouch or other suitable closures or sealing materials. Raised portion 2 is the surface utilized during heat-sealing. As a result, the sealing means can also function to maintain the association of the first and second container portions 11, 12 thereby obviating the need for first and second container portions 11, 12 to be attached to one another. However, if it is desirable, first and second container portions 11, 12 can be attached to one another by, for example, a hinge, so long as it is possible to separate first and second container portions 11, 12 sufficiently to permit second container portion 12 to accomplish its function of transferring the packaged device from container 10 to another location.

Typically, container 10 is constructed of plastic material. The material of container 10 may be sterilizeable, particularly if the packaged device is a sterile device, such as a medical device. Container 10 is also preferably lightweight, and inexpensive to manufacture. First and second container portions 11, 12 may be constructed of the same or different materials. If the device to be shipped in container 10 is radioactive, for example, it may be desirable to construct all or a portion of container 10 from a radiation shielding material, or to include a radiation shielding material in all or a portion of one or both of first and second container portions 11, 12. Container 10 may be sterilized and subsequently sealed, by, for example, a flexible material such as a pliable film such as a polyolefin film, shrink-wrap material, Tyvek®, flexible sheet material, or other suitable material prior to shipping. Alternatively, the device located inside of container 10 may be sterilized and subsequently sealed by, for example, a flexible material such as a pliable film, e.g. polyolefin film, shrink-wrap material, Tyvek®, flexible sheet material, or other suitable material, prior to closing the device in container 10. Container 10 may be sealed for the purpose of maintaining a sterile environment within container 10 during storage and shipping, or for the purpose of containing potentially hazardous materials such as corrosives, irritants, poisons or radioactive materials inside container 10 during storage and shipping. Upon arrival at its destination the sealing material may be removed from container 10 in order to allow access to the contents of container 10.

First container portion 11 may include a peripheral flange 4 around all or a portion of the periphery of first container portion 11. Peripheral flange 4 can provide stability to container 10 when placed on a flat surface and may also be used to facilitate location of container 10 in a larger storage or shipping container designed for storage or shipment of a plurality of containers 10 at one time. Peripheral flange 4 may be provided with one or more protrusions 3 on the top surface thereof, which protrusions 3 can form part of a snap-fit or friction-fit engagement with another container or device, for example. Protrusions 3 can also be used to create a space between a lid, used to cover container 10, and container portion 11 to facilitate removal of the lid by permitting a person who is opening container 10 to get his/or her finger under the lid. Peripheral flange 4 may also include a raised portion 2 that extends around all or part of the periphery of first container portion 11 of container 10, and which is explained in greater detail below in reference to FIG. 4.

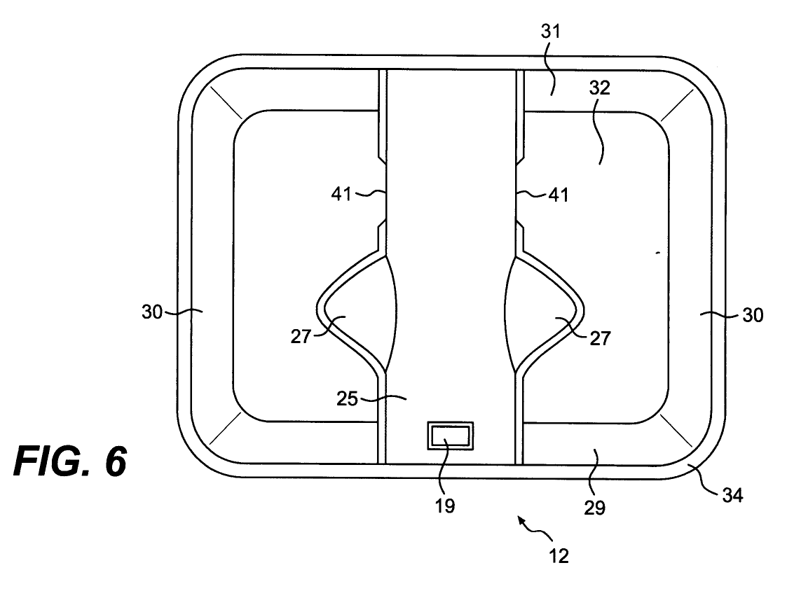

Referring to FIG. 6, second container portion 12 includes two sides 30, a front 29, a back 31 and a bottom 32 which together form a barrier or shield between the packaged device, when held in second container portion 12, and the hand of a user grasping second container portion 12 on the side opposite where the packaged device is held. The barrier or shield minimizes contamination of the packaged device by the user, or contamination of the user by the packaged device, during handling of the packaged device while held in second container portion 12 by providing a barrier between the user and the packaged device. Second container portion 12 may also be provided with a peripheral flange 34 that extends around all or a portion of the periphery of second container portion 12.

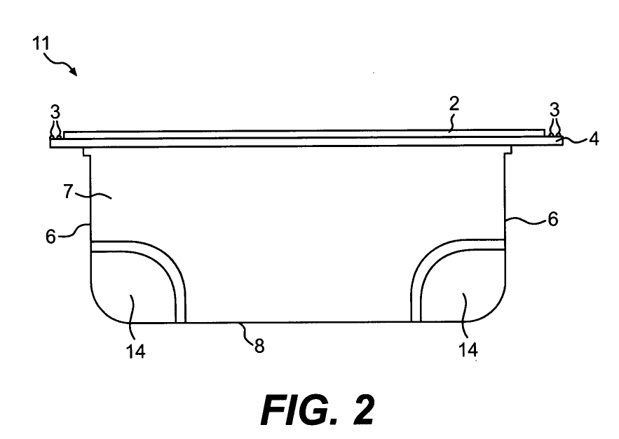

Referring to FIG. 2, there is shown a rear view of first container portion 11. In FIG. 2, the corner indentations 14 at each corner between bottom 8, sides 6 and back 7 of first container portion 11 can be clearly seen. Also shown in FIG. 2 is the presence of a pair of protrusions 3 located on each back corner of peripheral edge 4.

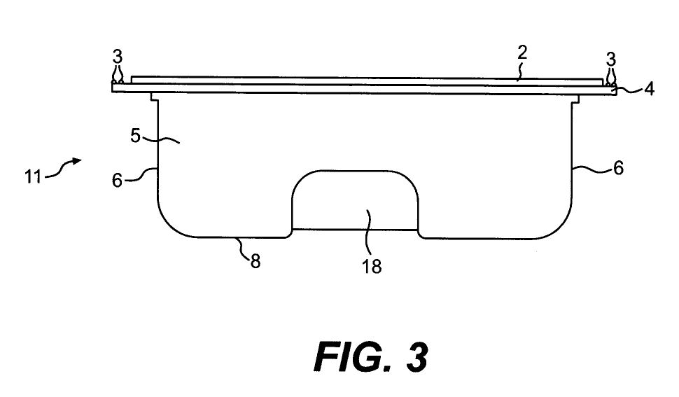

Referring now to FIG. 3, there is shown a front view of first container portion 11. In FIG. 3 it can be seen that the first container portion 11 includes a central indentation 18 located between bottom 8 and front 5 of the first container portion 11. Central indentation 18 forms a central ridge 20 in bottom 8 of first container portion 11, as best seen in FIG. 4. In this embodiment of container 10, central ridge 20 provides a surface 21 upon which a device packaged in container 10 may be positioned while remaining held by second container portion 12, as explained in greater detail below. Central ridge 20 further assists in supporting the packaged device during shipping.

It is not necessary to form shoulders 16 or central ridge 20 by the provision of indentations 14, 18, as in the present embodiment, but this type of structure has the advantage that it lends itself well to certain molding processes that facilitate production of container 10. Alternatively, first container portion 11 could be a circular or square container, or any other suitable shape, and include no indentations. In such a case, shoulders 16 and/or central ridge 20 could be provided, for example, by the location of additional structure within first container portion 11.

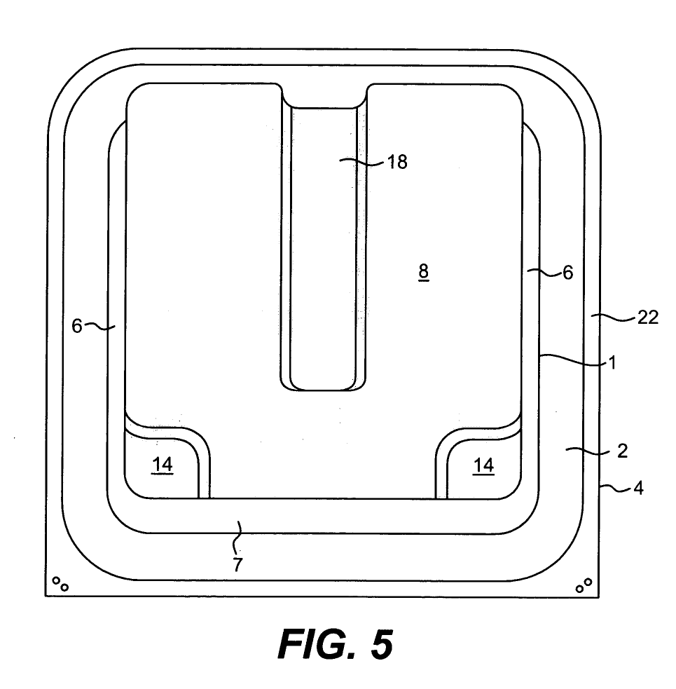

Referring to FIG. 4, which is a top, view of first container portion 11, the details of peripheral flange 4 can be seen. Specifically, peripheral flange 4 includes an outer rim 22, a raised portion 2 and an inner rim 23. Inner edge 1 of raised portion 2 defines an outer edge of inner rim 23. Inner rim 23 is sized to abut against a lower surface 33 of peripheral flange 34 of second container portion 12. In this manner, second container portion 12 can be held with its bottom 32 raised above bottom 8 and surface 21 of first container portion 11 by the abutment of lower surface 33 of peripheral flange 34 against inner rim 23 of peripheral flange 4 of first container portion 11. Raised portion 2 of peripheral flange 4 limits relative movement of second container portion 12 relative to first container portion 11 in the lateral direction by confining peripheral flange 34 of second container portion 12. FIG. 5 shows a bottom, isometric view of first container portion 11.

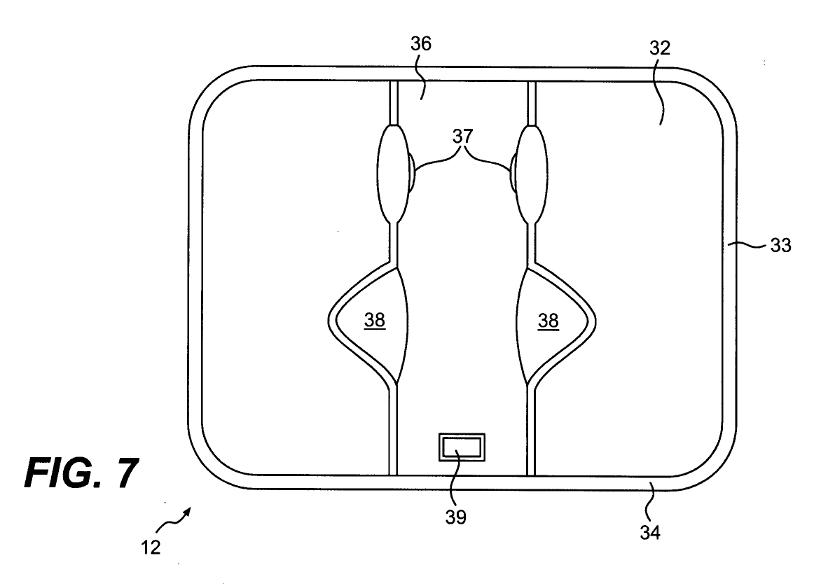

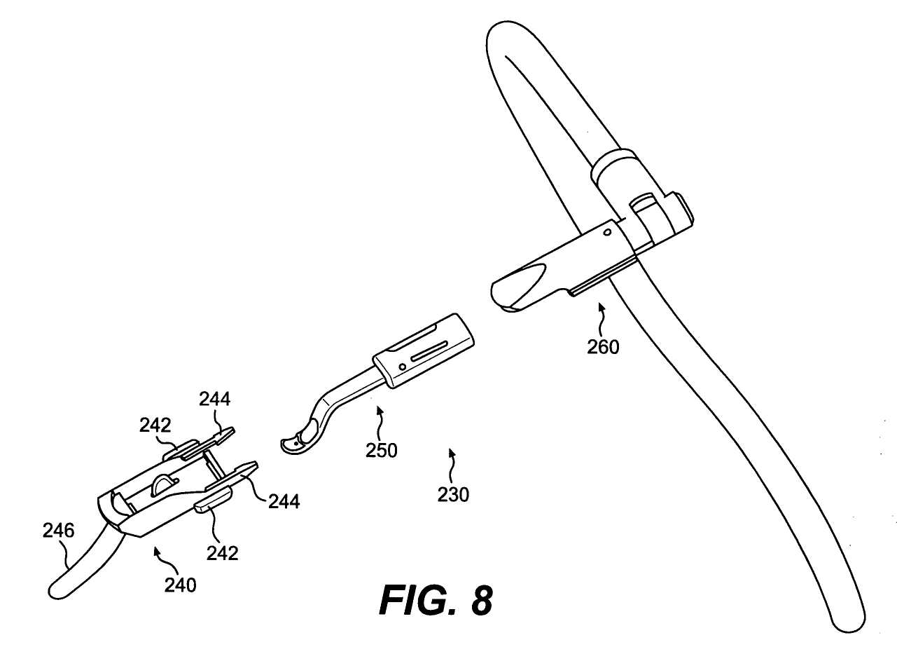

FIG. 6 shows a top view of second container portion 12. Front 29, sides 30, back 31 and bottom 32 together form second container portion 12. In the center of second container portion 12 there is a central cylindrical ridge 25 which forms a central cylindrical trough 36 in the outer surface of bottom 32 of second container portion 12, as shown in FIG. 7. Central cylindrical trough 36 may be shaped to accommodate the device that is being packaged in container 10, which in the example provided is the distal portion of an ocular brachytherapy applicator, such as that described in applicant's co-pending U.S. patent application Ser. No. 10/342,536, filed on Jan. 15, 2003, the disclosure of which is hereby incorporated by reference for the purpose of describing the details of the ocular brachytherapy applicator. An example of an ocular brachytherapy applicator 230 is shown in FIG. 8.

In this case, the distal portion 240 of the ocular brachytherapy applicator 230 is to be used in sterile condition and thus it is desirable to ship distal portion 240 in container 10. Distal portion 240 has a generally cylindrical shape and thus central cylindrical trough 36 is provided with a generally cylindrical shape to accommodate this. Central cylindrical trough 36 also includes a pair of recesses 38 which appear as bulges 27 in the top view of second container portion 12 shown in FIG. 6. Recesses 38 are provided for the purpose of accommodating the special shape of handles 242 of distal portion 240 of ocular brachytherapy device 230. In alternative embodiments, central cylindrical trough 36 may be replaced by a trough of any other suitable shape depending on the shape of the device intended for storage and shipment in container 10.

Central cylindrical trough 36 is one example of a holding structure that may take many different shapes, depending on the device to be packaged. The function of the holding structure is to hold the packaged device so that second container portion 12 may be grasped and used to transfer the packaged device from container 10 to another location without having to touch the packaged device directly. Thus, in certain embodiments, the holding structure may be designed to provide a snap-fit, form-fit or friction-fit with the packaged device. Alternatively, the holding structure may provide a close fit with the packaged device and the second container portion 12 may be fabricated from a flexible material which allows sufficient deformation of the holding structure, by application of force from the user's hand, to grasp and hold the packaged device in the holding structure. Other ways of releasably attaching the packaged device to the container 10 may also be employed. For example, a hook and loop attachment such as Velcro® or another similar attachment can be employed. Other possible holding structures include, for example, releasable clips, magnets, and weak adhesives. Optionally, the holding structure may have one or more textured surfaces in order to increase friction between the holding structure and the packaged device.

In general, it is desirable that the packaged device be firmly retained by the holding structure to facilitate transfer of the packaged device from the package to another location without the risk of dropping the packaged device. Once the packaged device is at the desired location, however, it should be simple and easy to then detach the packaged device from the holding structure, preferably without the user making direct contact with the packaged device. Accordingly, any releasable attachment structure that fulfills these functional goals, can be used in the various embodiments of the container of the present invention.

In the embodiment shown in FIGS. 6-7, central cylindrical trough 36 is provided with a pair of opposing protrusions 37 formed as part of second container portion 12 which provide a snap-fit or friction-fit with the ocular brachytherapy device. Since the contact area between protrusions 37 and the ocular brachytherapy device is small, removal of the device from second container portion 12 is facilitated because relatively little force will be required to deform protrusions 37 sufficiently to remove the packaged device. Another advantage of employing protrusions 37 is that corresponding recesses 41 are formed in the opposite surface of second container portion 12. Recesses 41 provide one example of a grip for gripping second container portion 12 between two fingers of a single hand for handling second container portion 12.

Second container portion 12 may include a variety of different grips to facilitate gripping of second container portion 12 to transfer the packaged device from container 10 to another location. For example, the holding structure can be shaped to receive the packaged device and provide a grip on the surface of second container portion 12 opposite the holding structure. Alternatively, one or more depressions or recesses of any suitable shape for facilitating gripping of second container portion 12 by one or more fingers can replace recesses 41. In another embodiment, the grip may be in the form of one or more protrusions that can interact with one or more of the user's fingers or the user's hand for facilitating gripping of second container portion 12. In a still further embodiment, the grip may be a pair of flat surfaces that are aligned so that two fingers can be used to pinch against the flat surfaces and hold second container portion 12. Also, combinations of two or more different types of grips can be employed.

Second container portion 12 may also include an orienting mechanism for facilitating placement of the packaged device in second container portion 12 with the device oriented in a particular orientation. In the embodiment shown in FIGS. 6-7, an orienting mechanism is provided by a rectangular depression 39 in central cylindrical trough 36, as shown in FIG. 7, which appears as a rectangular bulge 19 on the outside of central cylindrical ridge 25, as shown in FIG. 6. Rectangular depression 39 is designed to receive a protruding portion of the packaged device such that when the packaged device is properly oriented in second container portion 12, the protruding portion of the packaged device will be located in rectangular depression 39. In this manner, the packaged device can be oriented relative to second container portion 12 to facilitate handling of the packaged device using second container portion 12.

A variety of different structures can serve as viable orienting mechanisms within the scope of the present invention. For example, the holding structure can be shaped to receive the packaged device only if it is inserted in the proper orientation. Alternatively, rectangular depression 39 can be replaced by a suitable depression of any shape that complements the shape of a structure on the packaged device that can be used to orient the device in the package. In another embodiment, the orienting mechanism may be a protrusion that fits into a corresponding recess in the packaged device. In a still further embodiment, the orienting mechanism may be a line or other mark which is to be aligned with a corresponding mark or structure on the packaged device to thereby ensure the proper orientation of the packaged device. Also, combinations of two or more orienting mechanisms can be employed.

In alternative embodiments, peripheral flange 34 may include releasable attachment members, not shown, to releasably secure second container portion 12 to first container portion 11. The releasable attachment members may be in the form of physical components that interlock, such as a slot and peg mechanism, or peripheral flange 32 of second container portion 12 may engage slits formed in, for example, raised portion 2 of peripheral flange 4 of first container portion 11. Also, snap-fit, friction-fit, releasable adhesive and/or hook and loop attachments, as well as other suitable, conventional releasable attachments, can be employed.

Other types of devices can be packaged in container 10. For example, brachytherapy devices, probes, knives, scissors, cutters, applicators, catheters, and other devices used in sterile environments may also be packaged in various embodiments of container 10 of the present invention. Also, hazardous devices may be packaged in container 10. A skilled person would be able to adapt container 10 to a particular device, using the present disclosure, by providing a suitable holding structure in second container portion 12. Container 10 could also be used for holding hazardous objects where directly grasping with the hands would be undesirable, such as, for example, devices that may include corrosive, irritant, poisonous or radioactive material, or other similarly hazardous materials. In this case, the barrier of second container portion 12 could act as a physical barrier to prevent contact with corrosive, irritant, or poisonous material, or as a radiation barrier to prevent transmission of radiation from the packaged device to the user.

Referring to FIG. 8, there is shown an ocular brachytherapy device 230 which includes a distal portion 240, which must be used in sterile condition, an applicator portion 250 and an articulating arm attachment portion 260. Container 10 of the present invention is designed for packaging, shipment and deployment of distal portion 240 of ocular brachytherapy device 230. As can be seen in FIG. 8, distal portion 240 includes a pair of handles 242 for which provision is made in container 10 by the inclusion of recesses 38. Distal portion 240 also includes a pair of prongs 244 which extend from the proximal end of distal portion 240 for the purpose of attaching distal portion 240 to articulating arm attachment portion 260. Thus, container 10 is designed to permit prongs 244 to extend beyond the periphery of second container portion 10 when distal portion 240 is held by second container portion 10 so that distal portion 240 can be manipulated to attach it to articulating arm attachment portion 260.

Distal portion 240 also includes a flexible, sterile sheath 246 extending from the distal end of distal portion 240, as shown. Flexible, sterile sheath 246 can be tucked under distal portion 240 for packaging in order to reduce the size of the container 10 required to house distal portion 240. For this purpose, container 10 may be designed to suspend second container portion 12 and the body of distal portion 240 above the bottom 8 of first container portion 11 when first and second container portions 11, 12 are associated with one another to allow some space for sterile sheath 246 to be tucked under distal portion 240 when located in container 10.

In a second aspect, the present invention provides a method for shipping devices that minimizes contamination of the devices during shipping and transfer of such devices out of the shipping container for use. In the method, a container is provided having first and second container portions. The second container portion is provided with a grip that is graspable by a user's hand and a holder for holding the device using that portion of the container. In the method, the device is first placed into the container. Then, upon opening the container, the user grasps the grip that forms part of the second container portion and removes the device from the container using the second container portion to manipulate the device. For example, the device can be attached to an articulating arm while held by the second container portion. In the final step of the method, the device is detached from the second container portion.

The method of the present invention may further include the step of orienting the device relative to the second container portion when placing the device in the second container portion. Holding the device in a particular orientation with the second container portion may facilitate manipulation of the device when it is transferred from the container to its point of use.

It is to be understood that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

1. A container for shipping and handling a device, said container comprising:
a first container portion; and
a second container portion which, when associated with said first container portion, substantially encloses said device; wherein said second container portion comprises a holding structure for holding said device in said second container portion, and a grip on an opposite side of said second container portion from said holding structure.

2. The container of claim 1, wherein said first container portion is of sufficient size to house said device when held in said second container portion.

3. The container of claim 1, wherein said first and second container portions are sterilizeable.

4. The container of claim 1, wherein said holding structure comprises a structure that provides an interaction with said device selected from the group consisting of a snap-fit, friction-fit, a form-fit, and a releasable attachment.

5. The container of claim 4, wherein said holding structure is selected from the group consisting of an adhesive, a hook and loop attachment, one or more clips, two or more protrusions and combinations of two or more of these structures.

6. The container of claim 3, further comprising a third container portion which seals an interior of said container formed by said first and second container portions, from the environment.

7. The container of claim 4, wherein said grip comprises a structure selected from the group consisting of one or more recesses, one or more protrusions, one or more flat surfaces and combinations thereof.

8. The container of claim 7, wherein said first container portion is sized, relative to said second container portion, to house said device when held in said second container portion with at least part of said device extending beyond a periphery of said second container portion.

9. The container of claim 8, wherein said second container portion further comprises an orienting mechanism for ensuring that said device is oriented in a specific orientation relative to said second container portion.

10. The container of claim 9, wherein said device is selected from the group consisting of: brachytherapy devices, probes, knives, scissors, cutters, applicators, catheters and hazardous devices.

11. The container of claim 10, wherein the orienting mechanism is a structure that interacts with structure of the device to thereby orient the device relative to the container.

12. The container of claim 11, wherein the hazardous devices are selected from the group consisting of devices which comprise one or more of corrosive material, poisonous material, irritant material and radioactive material.

13. A method for packaging and deployment of a device, said method comprising the steps of:
providing a packaging container for said device,
locating said device in said packaging container in a manner whereby said device is releasably retained by one portion of said container,
grasping said portion of said container in which said device is releasably retained, transferring said device to another location while said device is releasably retained in said portion of said container, and
removing said device from said portion of said container.

14. A method as claimed in claim 13, wherein said placing step comprises the step of orienting said device relative to said portion of said container in which said device is releasably retained.

15. A method as claimed in claim 14, wherein said transferring step comprises the step of manipulating said device so as to interact with another device.

16. A method as claimed in claim 15, wherein said manipulating step comprises attaching said device to another device.

17. A container for shipping and handling a sterilizeable device in sterile condition, said container comprising:
a first container portion;
a second container portion sized to fit at least substantially within said first container portion, and which, when associated with said first container portion, substantially encloses said device; wherein said second container portion comprises a holding structure for holding said device in said second container portion, and a grip on an opposite side of said second container portion from said holding structure; and
a third container portion which seals said first and second container portions to provide an airtight environment in said container.

18. A container as claimed in claim 17, wherein said holding structure comprises a part of said second container portion sized to provide a fit with said device selected from the group consisting of a friction-fit, a snap-fit, a form-fit and a close-fit, and, when said fit is a close fit, said second container portion is sufficiently flexible to permit sufficient deformation of said holding structure to grasp and hold said device during transfer of said device from said container to another location.

19. A container as claimed in claim 18, wherein said grip comprises a part of said second container portion shaped to facilitate grasping of said second container portion by one or more fingers of a human hand.

20. A container as claimed in claim 19, wherein said first container portion is sized, relative to said second container portion, to house said device when held in said second container portion with at least part of said device extending beyond a periphery of said second container portion.

21. The container of claim 17, wherein said second container portion further comprises an orienting mechanism for ensuring that said device is oriented in a specific orientation relative to said second container portion.

22. The container of claim 21, wherein said device is selected from the group consisting of: brachytherapy devices, probes, knives, scissors, cutters, applicators, catheters and hazardous devices.

23. The container of claim 22, wherein the orienting mechanism is a structure that interacts with structure of the device to thereby orient the device relative to the container.

24. The container of claim 23, wherein the hazardous devices are selected from the group consisting of devices which comprise one or more of corrosive material, poisonous material, irritant material and radioactive material.

25. A method for packaging and deployment of a sterilizeable device in sterile condition, said method comprising the steps of:
providing a packaging container for said sterile device,
locating said device in said packaging container in a manner whereby said device is releasably retained by one portion of said container,
grasping said portion of said container in which said device is releasably retained,
transferring said device to another location while said device is releasably retained in said portion of said container, and
removing said device from said portion of said container.

26. A method as claimed in claim 25, wherein said placing step comprises the step of orienting said device relative to said portion of said container in which said device is releasably retained.

27. A method as claimed in claim 26, wherein said transferring step comprises the step of manipulating said device so as to interact with another device.

28. A method as claimed in claim 27, wherein said manipulating step comprises attaching said device to another device.
